(19) United States
(12) Patent Application Publication
(10) Pub. No.: US 2024/0261305 A1
(43) Pub. Date: Aug. 8, 2024

(54) OILY FORMULATIONS OF CANNABINOIDS

(71) Applicant: VELLEJA RESEARCH S.R.L., Pontenure (PC) (IT)

(72) Inventor: Francesco Di Pierro, Pontenure (PC) (IT)

(73) Assignee: VELLEJA RESEARCH S.R.L., Pontenure (PC) (IT)

(21) Appl. No.: 18/565,715

(22) PCT Filed: Jun. 3, 2022
(86) PCT No.: PCT/IB2022/055183

§ 371 Date: Nov. 30, 2023

(30) Foreign Application Priority Data
Jun. 8, 2021 (IT) 102021000014909

(51) Int. Cl. A61K 31/00; A61K 8/9789; A61K 36/21; A61K 45/06

(52) U.S. Cl. CPC A61K 31/658; A61K 8/9789; A61K 36/21; A61K 36/3482; A61K 45/06; A61K 2800/10

(57) ABSTRACT

The invention relates to cannabinoid solutions in a mixture of sesame oil, amaranth oil and squalene. The invention also relates to pharmaceutical or nutraceutical formulations comprising said solutions.

The invention relates to cannabinoid solutions in a mixture of sesame oil, amaranth oil and squalene. The invention also relates to pharmaceutical or nutraceutical formulations comprising said solutions.

### STATE OF THE ART

Cannabinoids are a group of compounds derived from Cannabis sativa L., commonly known as “marijuana” and used for many centuries as a drug or therapeutic agent (Nocerino E, Amato M, Izzo AA. Cannabis and cannabinoid receptors. Phytotherapy 2000; 71: S6-S12).

The use of cannabis has been subject to legislative restrictions due to the psychotropic effects linked to the presence of delta-9 tetrahydrocannabinol (THC). However, other cannabinoids extracted from Cannabis have no psychotropic effect: among these, tetrahydrocannabinolic acid (THCA), cannabinolic acid (CBNA), cannabidiolic acid (CBDA) and cannabigerolic acid (CBGA), and the decarboxylated derivatives cannabinol (CBN), cannabidiol (CBD) and cannabigerol (CBG).

The pharmacological activity of cannabinoids is mediated by interaction with CB1 and CB2 receptors (Mackie K. Annu Rev Pharmacol Toxicol 2006; 46:101-22; Reggio P H. Current Pharmaceutical Design 2003; 9:1607-33). The CB1 receptor is mainly expressed in the central nervous system while the CB2 receptor is mainly expressed in the immune system and in haematopoietic or blood cells.

The most interesting therapeutic applications of cannabinoids include analgesia, alleviation of nausea and vomiting during chemotherapy, anti-rheumatic and antipyretic properties, asthma, activity on intestinal motility and appetite regulation (Lambert D M et al., Journal of Medicinal Chemistry 2005; 48: 5059-87).

Despite their therapeutic potential, only two cannabinoid receptor agonists, dronabinol and nabilone, and a cannabinoid receptor antagonist, Rimonabant, authorised for the treatment of obesity, are currently available for medical use in some countries, but not in Italy. Finally, a natural cannabis extract, marketed under the name Sativex®, is used in many countries for the symptomatic treatment of neuropathic pain in multiple sclerosis. Sativex® is formulated as an oromucosal absorption spray and contains approximately equal amounts (1:1) of dronabinol (THC) and cannabidiol (CBD).

The use of cannabidiol as an antiepileptic requires very high doses (up to 1 gram/day in children) and constant consumption. There is convincing evidence that in vivo CBD is not converted to THC, but the processes of obtaining it include reactions that can potentially generate THC, or use biomasses containing THC.

CBD has unfavourable pharmacokinetics and stability problems. Due to its lipophilic nature (Log P 6.3) (Odi R. et al., Epilepsia. 2020; 61:1543-1552.), CBD is commonly formulated in oil or alcohol vehicle. The oral bioavailability of CBD is estimated to be 6% (Millar S. A. et al., Front. Pharmacol. 2018; 9:1365). The time to reach peak plasma concentration after oral administration is slow (1-4 h), the Cmax after oral administration of 20 mg CBD is 2.4 ng/mL and the half-life is between 1.4 and 10.9 hours.

First-pass metabolism represents a significant barrier to CBD bioavailability (Taylor L. et al., J. Clin. Pharmacol. 2019; 59:1110-1119). In addition to being poorly bioavailable, CBD presents problems of instability being sensitive to temperature, light and oxidative processes. Sesame oil formulations are considered effective in promoting CBD bioavailability and stability (Silmore L H et al., Pharmacotherapy. 2021 April; 41(4):405-420).

U.S. Pat. No. 10,080,736 discloses microcapsule formulations of cannabinoids. U.S. Pat. No. 10,806,707 discloses capsules comprising cannabis oil and essential oils. WO2020/044118 discloses cannabinoid formulations in camelina oil possibly associated with fish and flax oils.

### DESCRIPTION OF THE INVENTION

It has now been found that sesame oil, combined with amaranth oil (both in winterized and non-winterized form), added with squalene (between 0.1 and 50% in the final blend) in the formulation of pure cannabidiol (either in amorphous form obtained by extraction or in crystalline form obtained by synthesis) or blended (1 to 99%) with other cannabinoids and cannabis terpenes, allows an important kinetic gain and a substantial improvement in the stability of CBD itself.

“Winterized form” means a form that has undergone winterization, i.e. fractional crystallization carried out to achieve separation of various fractions with different melting temperatures.

The invention, in a first aspect, therefore relates to solutions of cannabinoids, in particular cannabidiol and cannabigerol, in a mixture of sesame oil, amaranth oil and squalene.

In another aspect, the invention provides nutraceutical, pharmaceutical or cosmetic formulations comprising said solutions.

Cannabidiol can be used as a substantially pure compound or in the form of a hydro-ethanolic extract of Cannabis sativa free of delta-9-tetrahydrocannabinol with a 75-85% cannabidiol content.

A preferred extract has the following specifications:

| | |
|---|---|
| Colour: | Yellow-orange to dark red |
| | dark |
| | (Visual/Colorimetric) |
| Water activity: | <0.6 WA (dual sensor) |
| Transparency: | Transparent (Visual/Microscopic) |
| Particles: | absent (Visual/microscope) |
| Solubility: | Soluble in oils and alcohols |
| Chlorophylls: | <1% (Spectrophotometric) |
| Total sesquiterpenes | >1% GCMS |
| (sum of Beta-caryophyllene, | |
| alpha-humulene, nerolidol, | |
| caryophyllene oxide, | |
| guaiol, alpha-bisabolol): | |
| Total CBD (CBD + CBDA): | 75-85% (HPLC) |
| Delta-9-tetrahydrocannabinol: | not detected (HPLC/GCMS) |
| CBD/THC ratio: | >1000 (HPLC) |
| CBN: | <0.2% (HPLC) |
| CBG: | 0.8-1% (HPLC) |
| % decarboxylation: | >95% (total CBD/CBD) |

The weight ratio of sesame oil, amaranth oil and squalene in the solutions of the invention is not critical, but is in principle between 100:100:1 and 100:5:0.1, while the weight ratio of cannabinoids to the mixture of oils and squalene is between 10:1 and 1:10.

The formulations comprising the solutions of the invention may further contain antioxidants selected from vitamins, N-acetyl-cysteine, lipoic acid, CoQ10, omega-3 fatty acids, polyphenols.

Examples of formulations include capsules, tablets, sachets, sublingual sprays, gels, emulsions, ointments, transdermal patches for local release, and ready-made solutions or solutions to be reconstituted.

### Pharmacological Experimentation

Data obtained in mice and rats demonstrated an increase in oral bioavailability for CBD from 5-6%, when administered as an unformulated oil extract, to approximately 50 and 60% (in mice and rats respectively). The use of sesame oil alone produced a kinetic detection of 25-30% in both animal models.

With regard to stability, the studies underlying the present invention revealed, at room temperature and in the dark, a degradation of CBD at 3 months of 11 and 8% respectively when in pure form (both amorphous and crystalline) or when in products obtained by ethanolic extraction from cannabis. When CBD was solubilised in sesame oil or amaranth oil or in squalene alone, the instability did not show a statistically significant difference. When CBD was formulated in a mixture of sesame oil, amaranth and squalene (with the latter at least 0.1%), the degradation rates under the same conditions dropped to 4 and 3%, respectively.

The obtained results show that the use of mixtures of sesame oil, amaranth oil and squalene improves both the absorption and stability of CBD.

### Formulation Examples

1) Soft gelatine capsule

| Ingredient | Quantity | |
|---|---|---|
| Cannabis oil extract (50% CBD) | 250 | mg |
| Sesame oil | 100 | mg |
| Amaranth oil | 10 | mg |
| Squalene | 1 | mg |
| Vitamin E | 0.01 | mg |

| | |
|---|---|
| Other excipients | q.s. |

2) Soft gelatine capsule

| Ingredient | Quantity | |
|---|---|---|
| Cannabis oil extract (CBD 50%) | 400 | mg |
| Sesame oil | 50 | mg |
| Amaranth oil | 5 | mg |
| Squalene | 0.5 | mg |
| Omega 3 | 50 | mg |

| | |
|---|---|
| Other excipients | q.s. |

3) Oily drops for sublingual use:

| Ingredient | Quantity | |
|---|---|---|
| Cannabis oil extract (CBD 50%) | 100 | mg |
| Dietary triglycerides | 2.5 | mg |
| Sesame oil | 25 | mg |
| Amaranth oil | 2.5 | mg |
| Squalene | 0.25 | mg |
| Vitamin E | 3.0 | mg |

| | |
|---|---|
| Dispersant/emulsifier | q.s. |

4) Oily drops for oral/nasal vaporizers and suffumigation:

| Ingredient | Quantity | |
|---|---|---|
| Cannabis oil extract (CBD 50%) | 200 | mg |
| Essential oil of mint | 5 | mg |
| Essential oil of thyme | 0.1 | mg |
| Sesame oil | 10 | mg |
| Amaranth oil | 1 | mg |
| Squalene | 0.10 | mg |
| Vitamin E | 5.0 | mg |

| | |
|---|---|
| Dispersant/emulsifier | q.s. |

5) Gummy discus for oral use

| Ingredient | Quantity | |
|---|---|---|
| Cannabis oil extract (50%) | 100 | mg |
| Rubber base | 100 | mg |
| Levilite | 20 | mg |
| Talc F.U. | 20 | mg |
| Vegetable magnesium stearate | 18 | mg |
| Lacquer gum | 12 | mg |
| Xylitol | 250 | mg |
| Gum arabic | 6 | mg |
| Carnauba wax | 0.2 | mg |

6) Water-dispersible sachets

| Ingredient | Quantity | |
|---|---|---|
| Cannabis oil extract (50%) | 250 | mg |
| adsorbed with maltodextrins | | |
| Sucrose | 2265 | mg |
| Citric acid | 50 | mg |
| Vitamin C | 60 | mg |
| Silicon dioxide | 20 | mg |
| Aroma | 150 | mg |

| | |
|---|---|
| Other excipients | q.s. |

7) Effervescent sachets

| Ingredient | Quantity | |
|---|---|---|
| Cannabis oil extract (50%) | 250 | mg |
| adsorbed with maltodextrins | | |
| Citric acid | 500 | mg |
| Vitamin C | 120 | mg |
| Silicon dioxide | 400 | mg |
| Aroma | 150 | mg |
| Acesulfame K | 15 | mg |

| | |
|---|---|
| Other excipients | q.s. |

8) Gel for skin application:

| Ingredient | Quantity |
|---|---|
| Cannabis extract (50%) | 2% |
| Sodium hyaluronate | 0.5% |
| Vitamin E | 0.5% |
| Other excipients | q.s. |

1. Cannabinoid solutions in a mixture of sesame oil, amaranth oil and squalene.

2. Solutions according to claim 1 wherein the cannabinoids are selected from cannabidiol and cannabigerol.

3. Solutions according to claim 2 wherein the cannabidiol is in the form of a hydro-ethanolic extract of Cannabis sativa free of delta-9-tetrahydrocannabinol with a cannabidiol titre of 75-85%.

4. Solutions according to claim 1, wherein the ratio by weight of sesame oil, amaranth oil and squalene ranges between 100:100:1 and 100:5:0.1.

5. Solutions according to claim 1, wherein the weight ratio of cannabinoids to the mixture of oils and squalene ranges between 10:1 and 1:10.

6. Nutraceutical, pharmaceutical or cosmetic formulation comprising the solutions of claim 1.

7. Formulations according to claim 6 further comprising antioxidants selected from vitamins, N-acetyl-cysteine, lipoic acid, CoQ10, omega-3 fatty acids, polyphenols.

8. Formulations according to claim 6 in the form of capsules, tablets, sachets, sublingual sprays, gels, emulsions, ointments, transdermal patches for local release, ready-made solutions or solutions to be reconstituted.
